(19) United States
(12) Patent Application Publication
(10) Pub. No.: US 2023/0293503 A1
(43) Pub. Date: Sep. 21, 2023

(54) USE OF BERBERINE ANALOG AND JAK INHIBITOR IN TREATMENT OF INFLAMMATORY DISEASES OF GASTROINTESTINAL TRACT

(71) Applicants: ENNOVABIO (ZHEJIANG) PHARMACEUTICALS CO., LTD., Zhejiang (CN); SHANGHAI ENNOVABIO PHARMACEUTICALS CO., LTD., Shanghai (CA)

(72) Inventors: Jianyong SHOU, Shanghai (CN); Lei JIANG, Shanghai (CN); Xian JIN, Shanghai (CN); Shengyang LIU, Shanghai (CN); Xudong MAO, Shanghai (CN); Jianhua ZHANG, Shanghai (CN)

(21) Appl. No.: 17/907,394

(22) PCT Filed: Mar. 26, 2021
(86) PCT No.: PCT/CN2021/083421

§ 371 Date: Apr. 7, 2023

(30) Foreign Application Priority Data
Mar. 26, 2020 (CN) 202010225184.7

(51) Int. Cl. A61K 31/4375; A61K 31/519; A61P 1/12; A61P 29/00

(52) U.S. Cl. CPC A61K 31/4375; A61K 31/519; A61P 1/12; A61P 29/00

(57) ABSTRACT

The present invention relates to a strategy for combining a berberine analog with a JAK inhibitor in the treatment of inflammatory diseases of the gastrointestinal tract. Specifically, provided by the present invention is a pharmaceutical composition containing the berberine analog and the JAK inhibitor, and a use thereof in the treatment of inflammatory diseases of the gastrointestinal tract. The composition may exhibit an improved effect over the use of berberine or the JAK inhibitor alone.

### FIELD OF THE INVENTION

The present invention relates to the field of small molecule medicine, and in particular, the present invention provides a strategy of using the combination of berberine analogs and JAK inhibitors, or using berberine analogs in a formulation with JAK inhibitors for the treatment of gastrointestinal inflammatory diseases.

### BACKGROUND OF THE INVENTION

JAK inhibitors, such as tofacitinib have been approved in several countries including US for the treatment of certain patients suffering from moderate to severe active rheumatoid arthritis (RA). Recently, tofacitinib has been approved by the US FDA for the treatment of moderate to severe ulcerative colitis (UC). In patients with ulcerative colitis, there are systemic exposure mediated adverse events for tofacitinib, which are similar to those reported in RA patients. In clinical trials with tofacitinib, a large number of systemic exposure mediated adverse events were reported, including increased rates of severe infections, opportunistic infections, and malignancies; and laboratory abnormalities, such as lymphopenia, neutropenia, elevated liver enzymes, elevated lipids and elevated serum creatinine. Therefore, the approved US product XELJANZ/XEJANZ XR (trade name) carries a boxed warning detailing various safety risks, including the risk of serious infections and malignancies. Additionally, the European Medicines Agency voted against the marketing authorization of tofacitinib for RA due to its overall safety profile in 2013. Therefore, the development of next generation JAK inhibitor drugs with improved safety for the treatment of localized inflammatory diseases focuses on limiting the systemic exposure of JAK inhibitors. For example, the local exposure of tofacitinib in the gastrointestinal tract should be increased, meanwhile minimizing the systemic exposure of tofacitinib in the treatment of UC.

Berberine, also known as berberinum, is an isoquinoline alkaloid extracted from plants, such as Coptis chinensis. Berberine is well documented and has been used in traditional Chinese medicine for over a thousand years. It is of low bioavailability. Berberine is mainly used in the treatment of gastrointestinal diseases in clinical practice, such as diarrhea and intestinal infection. Studies in recent years have also found certain therapeutic potential of berberine in cardiovascular diseases as well as glucose and lipid metabolism regulation. Berberrubine is the major in vivo metabolite of berberine. Studies in animal models have shown that berberrubine has similar therapeutic effects to berberine in ulcerative colitis. However, up to date, there is no effective way to improve the therapeutic effect of berberine or its analogs in the art.

Inflammation bowel disease (IBD) mainly includes ulcerative colitis and Crohn's disease. These chronic intestinal inflammatory diseases have long-term disease course, recurrent attacks, and long-term inflammation which may raise the risk of cancers. The incidence rate of IBD has been on the rise in recent years. It is currently believed that the pathogenesis of IBD may relate to genetics, environment, immunity and microorganisms, but the exact etiology has not been discovered. Clinical treatments mainly utilize amino salicylic acid drugs, adrenal glucocorticoid drugs and immunosuppressors, all of which, however, have certain adverse effects such as gastrointestinal discomfort and anaphylaxes. In conclusion, there is in an urgent need of a treatment plan with fewer adverse effects and improved therapeutic effects for chronic inflammatory bowel disease. JAK inhibitors have been clinically proven to have good anti-inflammatory effects. Berberine analogs are capable of regulating the intestinal flora, protecting intestinal barrier function and regulating oxidative stress.

### SUMMARY OF THE INVENTION

The combination of JAK inhibitors and berberine analogs may achieve better effects on the treatment of gastrointestinal inflammatory diseases. At the same time, the combination of drugs may reduce the dose of a single drug, and may reduce the adverse effects related to the single drug in clinical practice.

The present invention provides a pharmaceutical combination of JAK inhibitor (preferably tofacitinib or SHR0302) and a berberine analog (preferably berberrubine), which uses JAK inhibitor (preferably tofacitinib) and a berberine analog (preferably berberrubine) to prepare a pharmaceutical composition or administered successively to achieve better therapeutic effects than the single use at the same dose.

In the first aspect of the present invention, a pharmaceutical composition is provided, which comprises:
(A) a therapeutically effective amount of the first active ingredient, wherein the first active ingredient is a berberine analog which has a structure selected from the group consisting of:

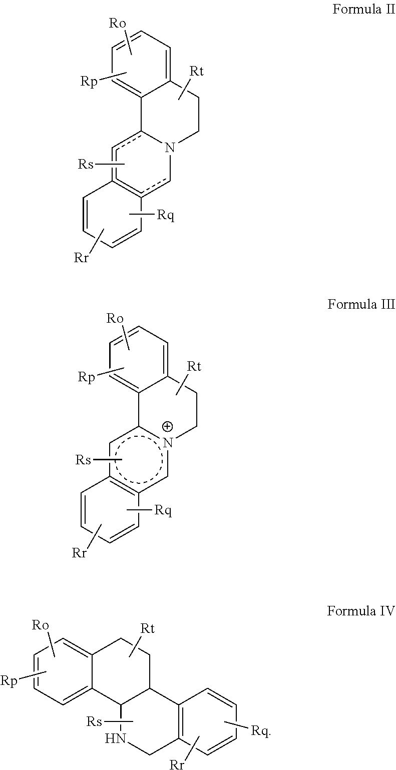

wherein,

dashed lines are chemical bonds or none;

Ro, Rp, Rq, Rr, Rs and Rt are each independently selected from the group consisting of H, hydroxy, substituted or unsubstituted C1-C4 alkyl, substituted or unsubstituted C1-C4 alkoxy;

or two of Ro, Rp, Rq, Rr, Rs and Rt on the same atom together form an oxygen atom;

or Ro, Rp, Rq, Rr, Rs and Rt on two adjacent atoms together with the atoms to which they are attached form a 5-7 membered heterocycle;

wherein the “substituted” means that one or more H atoms on a group are substituted by substituents selected from the group consisting of halogen, C1-C4 alkyl, and phenyl;
(B) a therapeutically effective amount of a second active ingredient, which is a JAK inhibitor;

and the mass ratio of the first active ingredient to the second active ingredient is in the range of 1-1000:1000-1.

In another preferred embodiment, the mass ratio of the first active ingredient to the second active ingredient is in the range of 1-10:10-1.

In another preferred embodiment, the berberine analog is selected from the group consisting of:

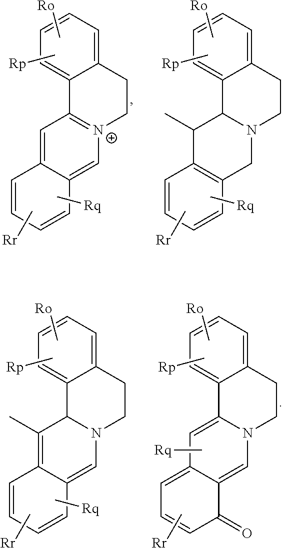

In another preferred embodiment, the berberine analog is selected from the group consisting of:

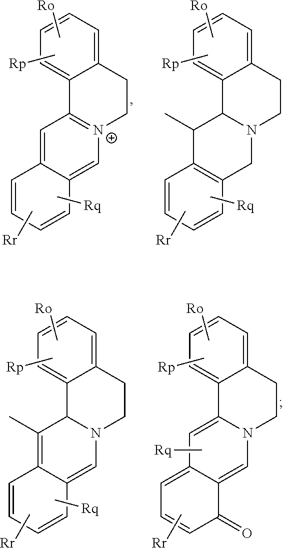

In another preferred embodiment, the berberine analog is selected from the group consisting of:

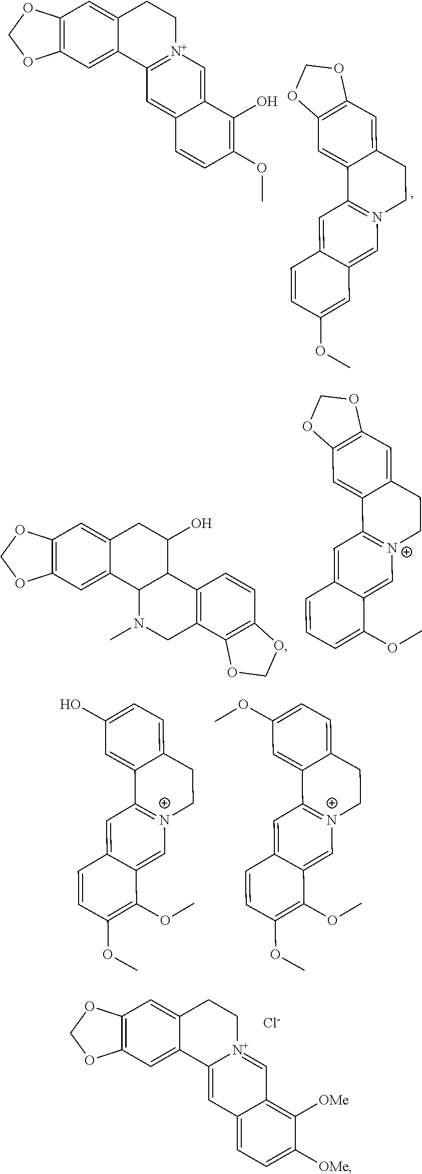

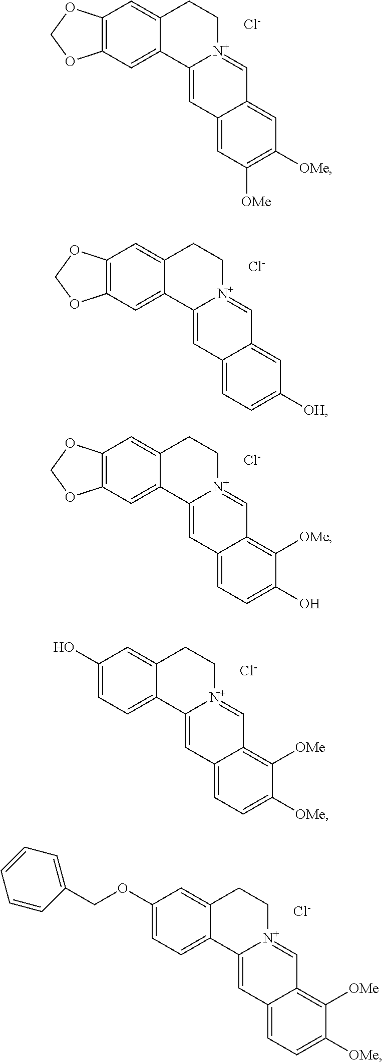

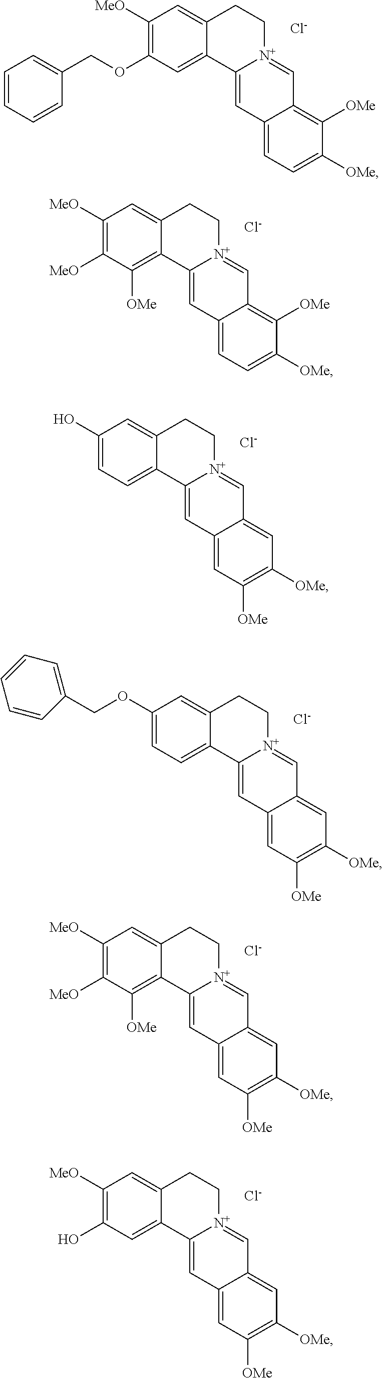

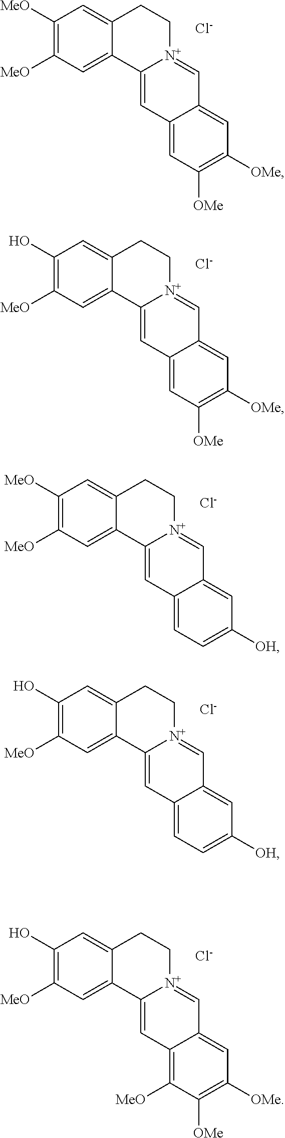

In another preferred embodiment, the JAK inhibitor can be selected from (but is not limited to) the group consisting of Tofacitinib, Ruxolitinib, Oclacitinib, Baricitinib, Peficitinib, Abrocitinib, Filgotinib, Upadacitinib, Delgocitinib, Itacitinib, Fedratinib, Decernotinib, SHR-0302, AZD-4205, ASN-002, BMS-986165, PF-06700841, PF-06651600, R-348, INCB-52793, ATI-501, ATI-502, NS-018, KL-130008, deuterium substituted JAK inhibitors, etc.

In another preferred embodiment, the JAK inhibitor is selected from the group consisting of:

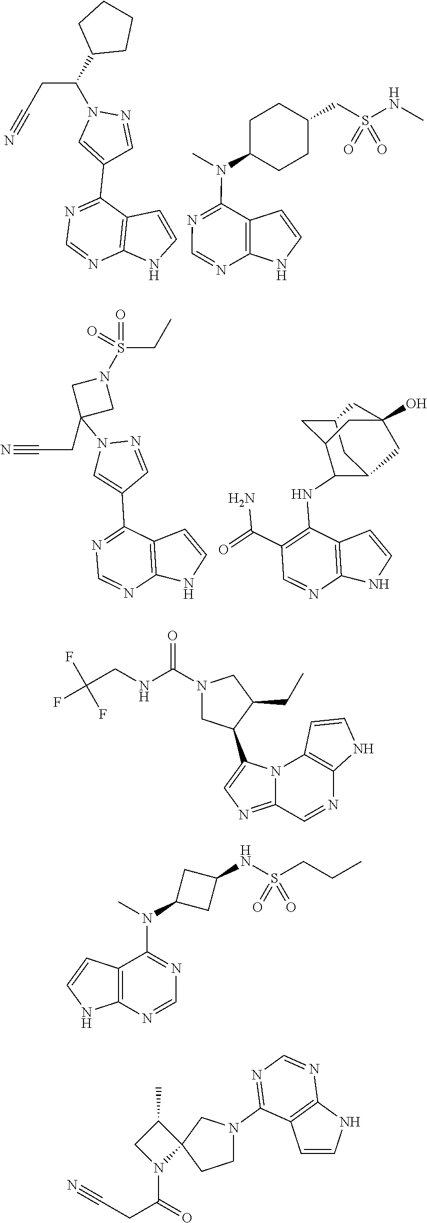

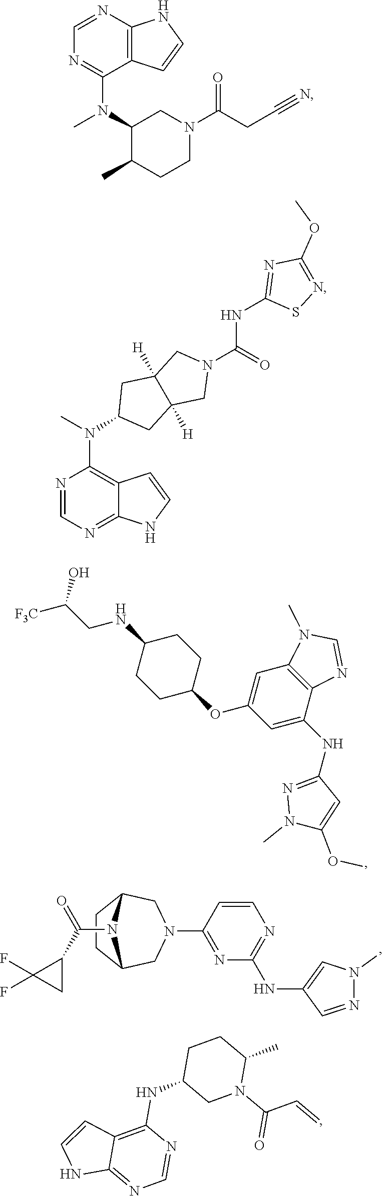

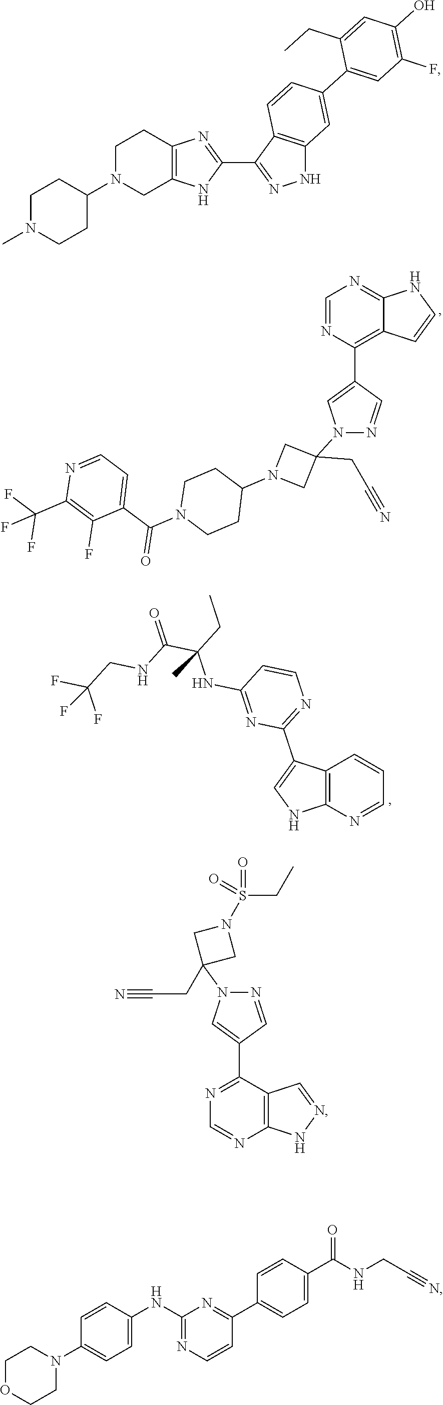 [SMILES: CCc1cc(O)c(F)cc1-c1ccc2c(-c3nc4c([nH]3)CN(C3CCN(C)CC3)CC4)n[nH]c2c1 ; N#CCC1(n2cc(-c3ncnc4[nH]ccc34)cn2)CN(C2CCN(C(=O)c3ccnc(C(F)(F)F)c3F)CC2)C1 ; CC[C@](C)(Nc1ccnc(-c2c[nH]c3ncccc23)n1)C(=O)NCC(F)(F)F ; CCS(=O)(=O)N1CC(CC#N)(n2cc(-c3ncnc4[nH]ncc34)cn2)C1 ; N#CCNC(=O)c1ccc(-c2ccnc(Nc3ccc(N4CCOCC4)cc3)n2)cc1]

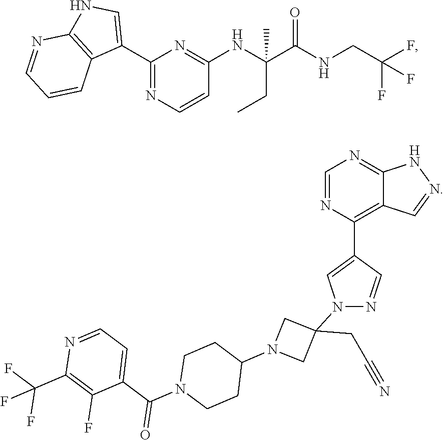 [SMILES: CC[C@@](C)(Nc1ccnc(-c2c[nH]c3ncccc23)n1)C(=O)NCC(F)(F)F ; N#CCC1(n2cc(-c3ncnc4[nH]ncc34)cn2)CN(C2CCN(C(=O)c3ccnc(C(F)(F)F)c3F)CC2)C1]

In another preferred embodiment, the pharmaceutical composition is an enteric-coated preparation.

In the second aspect of the present invention, a kit is provided, which comprises:
(A) a first formulation comprising a berberine analog;
(B) a second formulation comprising a JAK inhibitor; and
(C) an instruction for use;

and the berberine analog has a structure selected from the group consisting of:

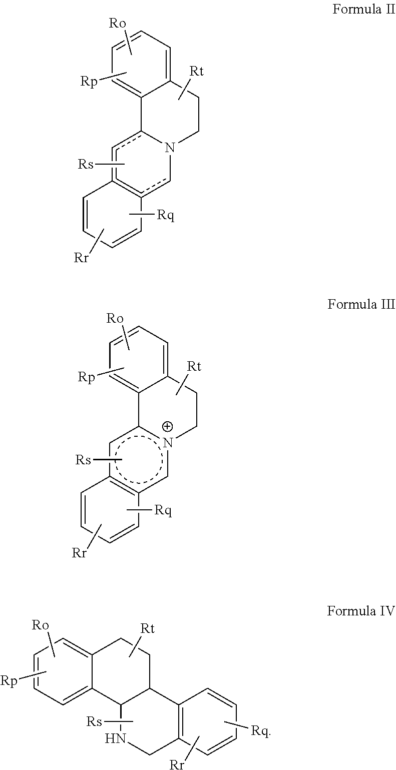

wherein,

dashed lines are chemical bonds or none;

Ro, Rp, Rq, Rr, Rs and Rt are each independently selected from the group consisting of H, substituted or unsubstituted C1-C4 alkyl, substituted or unsubstituted C1-C4 alkoxy;

or Ro, Rp, Rq, Rr, Rs and Rt on two adjacent atoms together with the atoms to which they are attached form a 5-7 membered heterocycle;

wherein the “substituted” means that one or more hydrogen atoms on a group are replaced with a substituent selected from the groups consisting of halogen, C1-C4 alkyl, and phenyl.

In the third aspect of the present invention, a combination of active ingredients is provided, which comprises following ingredients, or is composed of the following ingredients:
(A) a first active ingredient, wherein the first active ingredient is a berberine analog or a derivative thereof;
(B) a second active ingredient, which is a JAK inhibitor.

In the fourth aspect of the present invention, the use of the composition according to the first aspect of the present invention is provided, for the preparation of a pharmaceutical composition for the treatment of a disease selected from the group consisting of: Gastrointestinal inflammatory diseases (such as ulcerative colitis, Crohn's disease, colitis associated with immune checkpoint inhibitor therapy, collagenous colitis, lymphocytic colitis, pouchitis, acute/chronic gastritis, acute/chronic appendicitis), gastrointestinal autoimmune diseases (e.g., graft-versus-host disease, celiac sprue, autoimmune bowel disease), peptic ulcer, irritable bowel syndrome, gastric cancer, esophageal cancer, colon cancer.

It should be understood that in the present invention, each of the technical features specifically described above and below (such as those in the Examples) can be combined with each other, thereby constituting new or preferred technical solutions which need not be specified again herein.

### DESCRIPTION OF THE DRAWINGS

FIG. 1. Effects of tofacitinib, berberrubine, and the combination of tofacitinib and berberrubine on disease activity index (DAI) in an oxazolone-induced colitis model. The drug and dosage of each group are shown in the figure. Tofa: tofacitinib; BBR: berberrubine; Oxa: oxazolone.

FIG. 2. The combination of tofacitinib and berberrubine significantly reduced DAI AUC in the oxazolone-induced colitis model from day 1 to day 5, and the result of Bliss-independent model synergistic effect analysis is based on the mean value of each group. The drug and dosage of each group are shown in the figure. Tofa: tofacitinib; BBR: berberrubine; Oxa: oxazolone.

FIG. 3. Effects of JAK inhibitors SHR0302, berberrubine, and the combination of SHR0302 and berberrubine on disease activity index (DAI) in an oxazolone-induced colitis model. The drug and dosage of each group are shown in the figure. BBR: berberrubine; Oxa: oxazolone.

FIG. 4. The combination of SHR0302 and berberrubine significantly reduced DAI AUC in the oxazolone-induced colitis model from day 1 to day 5, and the result of Bliss-independent model synergistic effect analysis is based on the mean value of each group. The drug and dosage of each group are shown in the figure. BBR: berberrubine; Oxa: oxazolone.

### EMBODIMENTS FOR CARRYING OUT THE INVENTION

Through long-term and in-depth research, the inventors have found that using the combination of JAK inhibitor and a berberine analog would bring better therapeutic effects in the treatment of gastrointestinal diseases than using each single drug at the same dose. The present invention is completed on this basis.

### Terms

Unless otherwise defined, all technical and scientific terms used herein have the same meaning as commonly understood by one of ordinary skill in the art to which this invention belongs.

As used herein, the terms “containing” or “including (comprising)” may be an opened form, semi-closed form, or closed form. In other words, the terms also include situations, such as “essentially consisting of . . . ” or “consisting of . . . ”.

In the present application, the term “alkyl”, as a group or part of another group, means a fully saturated straight or branched hydrocarbon chain group which consists only of carbon atoms and hydrogen atoms, and has, for example, 1 to 12 (preferably 1 to 8, more preferably 1 to 6) carbon atoms, and is bonded to the rest of a molecule by a single bond, for example, including but not limited to, methyl, ethyl, n-propyl, isopropyl, n-butyl, isobutyl, sec-butyl, tert-butyl, n-pentyl, 2-methylbutyl, 2,2-dimethylpropyl, n-hexyl, heptyl, 2-methylhexyl, 3-methylhexyl, octyl, decyl and decyl. For the present invention, the term “C1-C6 alkyl” refers to an alkyl containing from 1 to 6 carbon atoms.

In the present application, the term “6-10 membered aromatic ring”, as a group or part of another group, means an aromatic ring having 6-10 ring atoms which are carbon atoms. The aromatic ring may be monocyclic or bicyclic, for example, a benzene ring, naphthalene ring, or the like.

In the present application, the term “C1-C4 alkoxy”, as a group or part of another group, means a straight-chain or branched alkoxy group having 1-4 carbon atoms; for example, methoxy, ethoxy, propoxy, isopropoxy, butoxy, isobutoxy, tert-butoxy, etc.

In the present application, the term “5-7 membered heterocycle”, as part of a group or other group, means a partially saturated or saturated heterocycle having 5-7 ring atoms, and at least 1 (which may be 1, 2 or 3) ring atom is a heteroatom selected from nitrogen, oxygen or sulfur. The heterocycle may be monocyclic or bicyclic, for example, pyrimidopyrazole ring, pyrazinoimidazole ring, pyridopyrazole ring, pyridoimidazole ring, pyridopyrimidine ring, and pyridopyridine ring.

### Compounds of the Present Invention

The compound of the present invention is a compound represented by formula I in the above, or a stereoisomer or racemate, or a pharmaceutically acceptable salt thereof.

The compound of the invention may contain one or more chiral carbon atoms and, thus, there may exist enantiomers, diastereomers, and other stereoisomeric forms. Each chiral carbon atom can be defined as (R)- or (S)-based on stereochemistry. The invention is intended to include all possible isomers, as well as racemic and optically pure forms thereof. Racemates, diastereomers or enantiomers may be employed as starting materials or intermediates of the preparation of the compounds of the invention. Optically active isomers can be prepared by chiral synthons or chiral reagents, or resolved using conventional techniques, such as by crystallization and chiral chromatography.

Conventional techniques for the preparation/isolation of individual isomers include chiral synthesis from a suitable optically pure precursor, or resolution of the racemate (or racemic form of a salt or derivative) using, for example, chiral high performance liquid chromatography. For example, see Gerald Gübitz and Martin G. Schmid (Eds.), Chiral Separations, Methods and Protocols, Methods in Molecular Biology, Vol. 243, 2004; A. M. Stalcup, Chiral Separations, Annu. Rev. Anal. Chem. 3:341-63, 2010; Fumiss et al. (eds.), VOGEL VOGEL 201002010 and Protocols, Methods in Molecular Biology, Vol. 243, 2004, 2004 Vol. 243, 20042004. Adverse reactions, such as gastrointestinal upset, allergic reactions, etc.; Heller, Acc. Chem. Res. 1990, 23, 128.

The term “pharmaceutically acceptable salt” includes pharmaceutically acceptable acid addition salts and pharmaceutically acceptable base addition salts.

“Pharmaceutically acceptable acid addition salt” means a salt formed with an inorganic or organic acid which retains the bioavailability of the free base without other side effects. Inorganic acid salts include, but are not limited to, hydrochlorides, hydrobromides, sulfates, nitrates, phosphates, and the like; and organic acid salts include, but are not limited to, formate, acetate, 2,2-dichloroacetate, trifluoroacetate, propionate, hexanoate, octoate, decanoate, undecylenate, glycolate, gluconate, lactate, sebacate, adipates, glutaric acid salts, malonates, oxalates, maleates, succinates, fumarates, tartrates, citrates, palmitates, stearates, oleates , cinnamate, laurate, malate, glutamate, pyroglutamate, aspartate, benzoate, methanesulfonate, besylate, p-toluenesulfonate, alginate, ascorbate, salicylate, 4-aminosalicylate, naphthalene disulfonate, and the like. These salts can be prepared by methods known in the art.

“Pharmaceutically acceptable base addition salt” means a salt formed with an inorganic or organic base which retains the bioavailability of the free acid without other side effects. Salts derived from inorganic bases include, but are not limited to, sodium salts, potassium salts, lithium salts, ammonium salts, calcium salts, magnesium salts, iron salts, zinc salts, copper salts, manganese salts, aluminum salts, and the like. Preferred inorganic salts are ammonium, sodium, potassium, calcium and magnesium salts. Salts derived from organic bases include, but are not limited to, following salts: primary amines, secondary amines and tertiary amines, substituted amines, including naturally substituted amines, cyclic amines, and basic ion exchange resins, for example, ammonia, isopropylamine, trimethylamine, diethylamine, triethylamine, tripropylamine, ethanolamine, diethanolamine, triethanolamine, dimethylethanolamine, 2-dimethylaminoethanol, 2-diethylaminoethanol, bicyclo hexylamine, lysine, arginine, histidine, caffeine, procaine, choline, betaine, ethylenediamine, glucosamine, methylglucosamine, theobromine, purine, piperazine, piperidine, N-ethylpiperidine, polyamine resin, and the like. Preferred organic bases include isopropylamine, diethylamine, ethanolamine, trimethylamine, dicyclohexylamine, choline, and caffeine. These salts can be prepared by methods known in the art.

The compounds described herein may contain one or more chiral centers. In such cases, the depiction or designation of a particular stereoisomer means that the indicated stereocenter has the indicated stereochemistry. It should be understood that, unless otherwise indicated, minor amount of other stereoisomers may also present, as far as the utility of the depicted or named compound is not eliminated by the presence of another stereoisomer.

### The First Active Ingredient

In the pharmaceutical composition or pharmaceutical combination of the present invention, the first active ingredient is a berberine analog, and the berberine analog has a structure selected from the group consisting of:

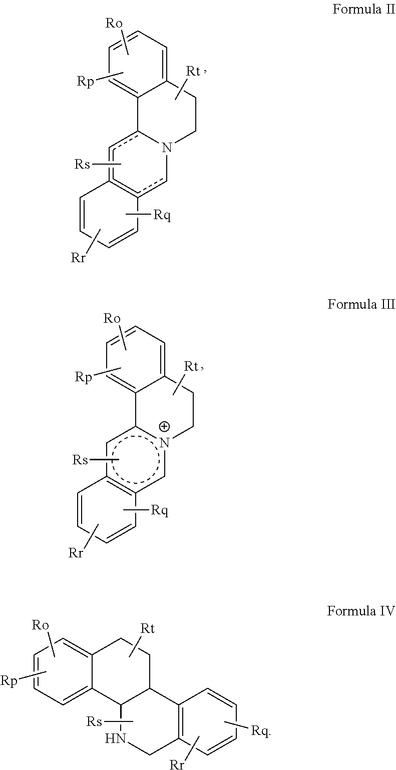

wherein,

dashed lines are chemical bonds or none;

Ro, Rp, Rq, Rr, Rs and Rt are each independently selected from the group consisting of H, hydroxy, substituted or unsubstituted C1-C4 alkyl, substituted or unsubstituted C1-C4 alkoxy;

or two of Ro, Rp, Rq, Rr, Rs and Rt on the same atom together form an oxygen atom;

or Ro, Rp, Rq, Rr, Rs and Rt on two adjacent atoms together with the atoms to which they are attached form a 5-7 membered heterocycle;

wherein the “substituted” means that one or more H atoms on the group are substituted by substituents selected from the group consisting of halogen, C1-C4 alkyl, and phenyl.

In another preferred embodiment, the berberine analog is selected from the group consisting of:

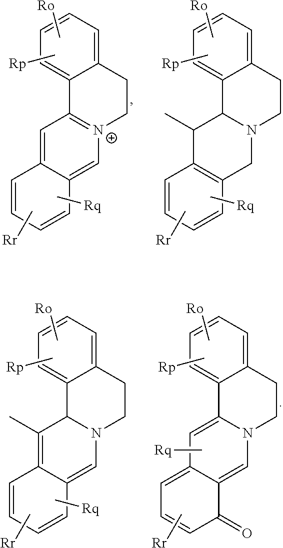

In another preferred embodiment, the berberine analog is selected from the group consisting of:

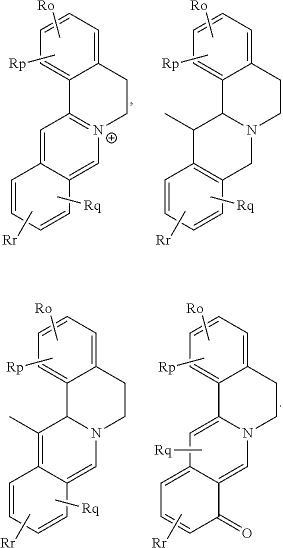

In another preferred embodiment, the berberine analog is selected from the group consisting of:

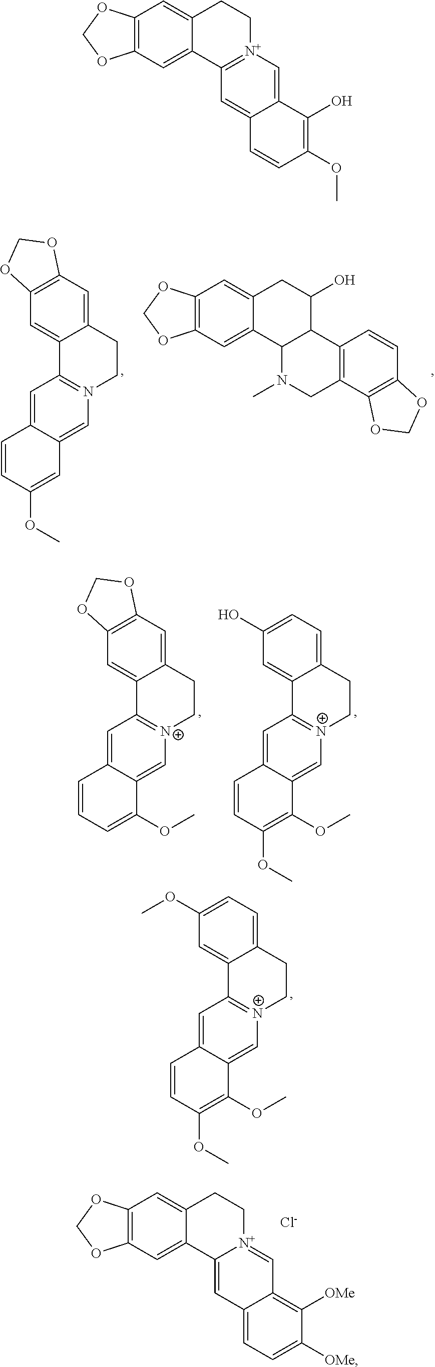

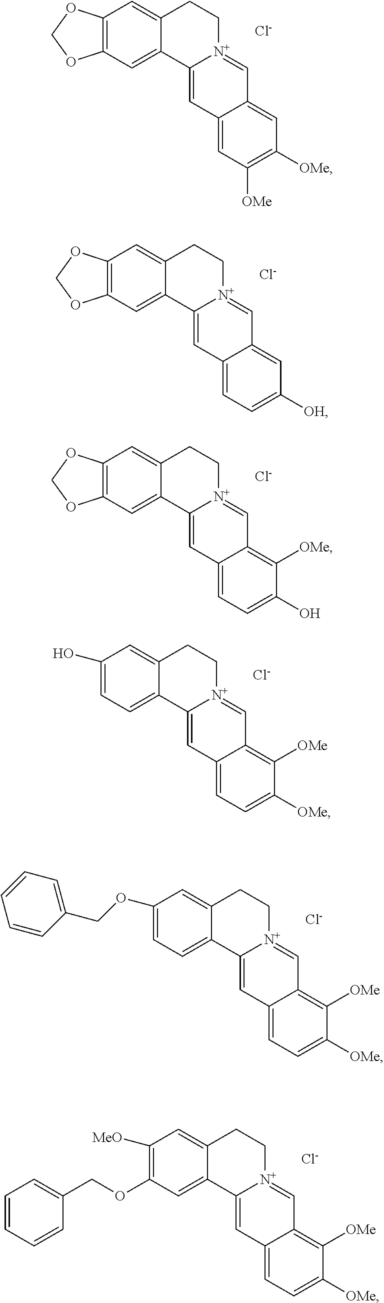

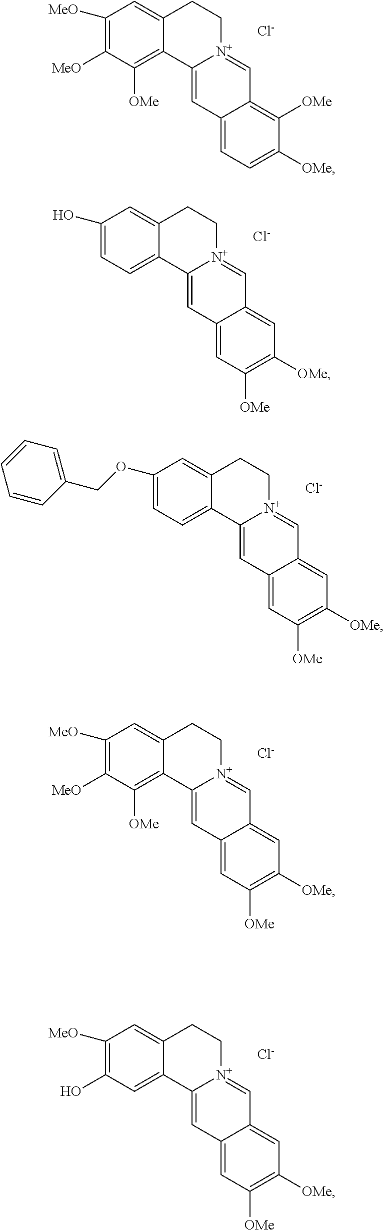

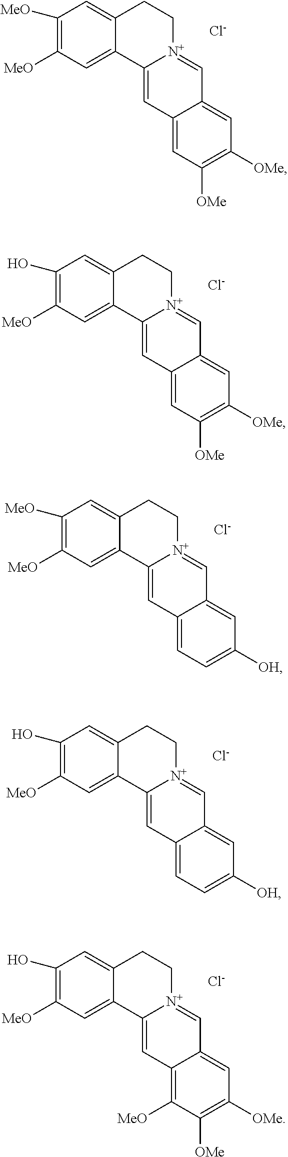

### The Second Active Ingredient

In the pharmaceutical composition or pharmaceutical combination of the present invention, the JAK inhibitor can be selected from (but is not limited to) the group consisting of Tofacitinib, Ruxolitinib, Oclacitinib, Baricitinib, Peficitinib, Abrocitinib, Filgotinib, Upadacitinib, Delgocitinib, Itacitinib, Fedratinib, Decernotinib, SHR-0302, AZD-4205, ASN-002, BMS-986165, PF-06700841, PF-06651600, R-348, INCB-52793, ATI-501, ATI-502, NS-018, KL-130008, deuterium substituted JAK inhibitors, etc.

In another preferred embodiment, the JAK inhibitor is selected from the group consisting of:

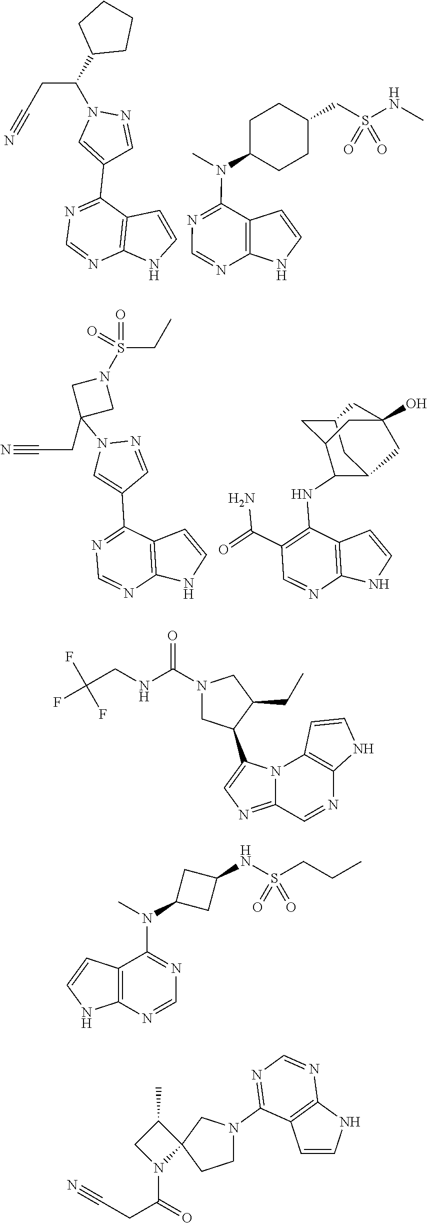

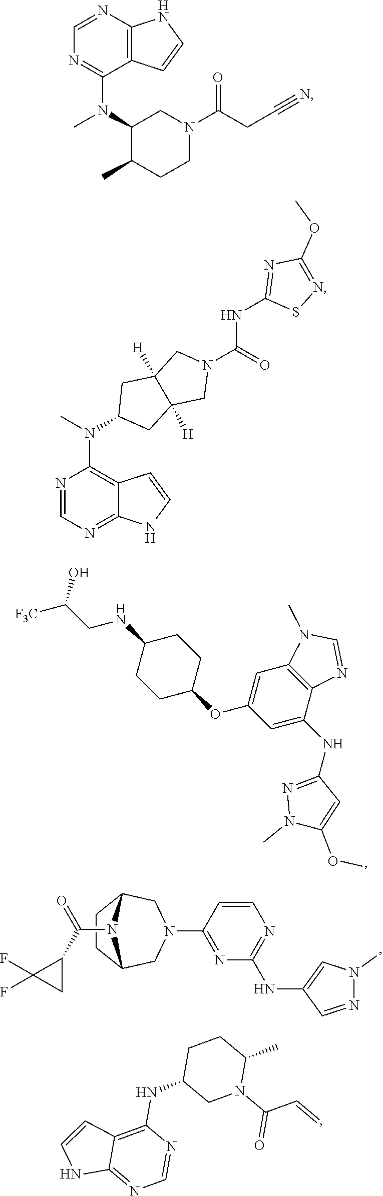

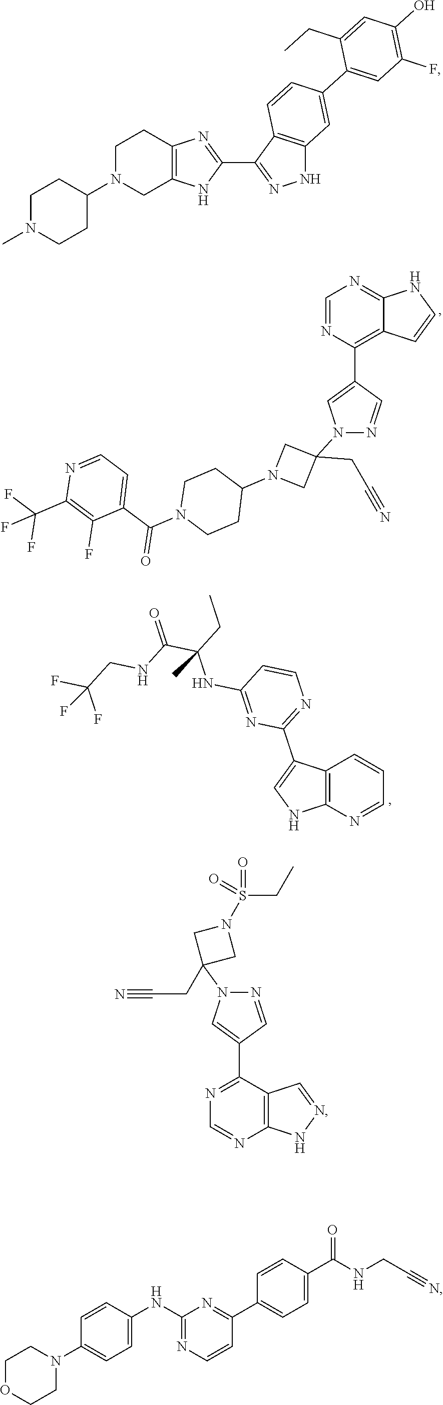 [SMILES: CCc1cc(O)c(F)cc1-c1ccc2c(-c3nc4c([nH]3)CN(C3CCN(C)CC3)CC4)n[nH]c2c1 ; N#CCC1(n2cc(-c3ncnc4[nH]ccc34)cn2)CN(C2CCN(C(=O)c3ccnc(C(F)(F)F)c3F)CC2)C1 ; CC[C@](C)(Nc1ccnc(-c2c[nH]c3ncccc23)n1)C(=O)NCC(F)(F)F ; CCS(=O)(=O)N1CC(CC#N)(n2cc(-c3ncnc4[nH]ncc34)cn2)C1 ; N#CCNC(=O)c1ccc(-c2ccnc(Nc3ccc(N4CCOCC4)cc3)n2)cc1]

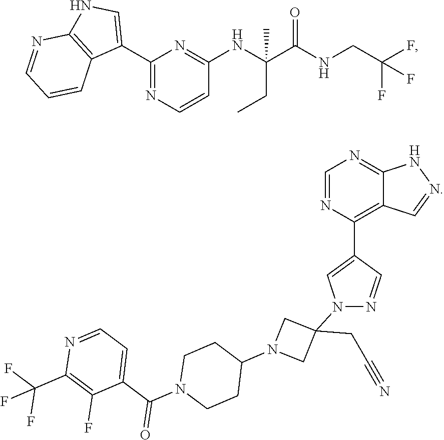 [SMILES: CC[C@@](C)(Nc1ccnc(-c2c[nH]c3ncccc23)n1)C(=O)NCC(F)(F)F ; N#CCC1(n2cc(-c3ncnc4[nH]ncc34)cn2)CN(C2CCN(C(=O)c3ccnc(C(F)(F)F)c3F)CC2)C1]

The mass ratio of the first active ingredient to the second active ingredient is 1-1000:1000-1. In another preferred embodiment, the mass ratio of the first active ingredient to the second active ingredient is 1-10:10-1.

### Application

Since the pharmaceutical composition of the present invention has excellent therapeutic effects on gastrointestinal inflammatory diseases, the compounds of the present invention and their various crystal forms, pharmaceutically acceptable inorganic or organic salts, hydrates or solvates thereof, as well as a pharmaceutical composition containing the compound(s) of the invention as the main active ingredient can be used for the prevention and/or treatment of intestinal functional diseases.

In the present application, the term “pharmaceutical composition” refers to a formulation of a compound of the invention and a medium generally accepted in the art for delivering a biologically active compound to a mammal, such as a human. The medium comprises pharmaceutically acceptable carriers. The purpose of the pharmaceutical composition is to promote the administration to an organism, thus facilitating the absorption of the active ingredients and thereby exerting the biological activity.

In the present application, the term “pharmaceutically acceptable” refers to a substance (such as a carrier or diluent) that does not affect the biological activity or properties of the compound of the invention, and is relatively non-toxic, i.e., the substance can be administered to an individual without causing undesirable biological reactions, or interacting with any of the components contained in the composition in an undesirable manner.

In the present application, the term “pharmaceutically acceptable excipients” includes, but is not limited to, any adjuvants, carriers, excipients, glidants, sweeteners, diluents, preservatives, dyes/colorants, flavoring agents, surfactants, wetting agents, dispersing agents, suspending agents, stabilizers, isotonic agents, solvents or emulsifiers approved by the relevant government authorities for the acceptable use in humans or domestic animals.

In the present application, terms “prevention”, “preventing” and “prevented” mean that the possibility of the occurrence or progression of a disease or condition in a patient is reduced.

In the present application, the term “treatment” and other similar synonyms include following meanings:
(i) preventing the occurrence of a disease or condition in a mammal, particularly when such a mammal is susceptible to the disease or condition, but has not been diagnosed as having the disease or condition;
(ii) inhibiting a disease or condition, i.e., inhibiting its development;
(iii) alleviating the disease or condition, i.e., degrading the condition of the disease or condition; or
(iv) relieving the symptoms caused by the disease or condition.

In the present application, the term “effective amount”, “therapeutically effective amount”, or “pharmaceutically effective amount” refers to an amount of at least one agent or compound that, after administration, is sufficient to alleviate one or more symptoms of the treated disease or condition to some extent. The result can be the reduction and/or alleviation of signs, symptoms or causes, or any other desired changes in a biological system. For example, an “effective amount” for treatment is an amount of a composition comprising a compound disclosed herein that is required to provide significant relieving effects on a condition in clinic. An effective amount suitable for any individual case can be determined using techniques such as dose escalation testing.

In the present application, the terms “taking”, “administering”, “applying” and the like refers to a method of delivering compound or composition to a desired site for biological action. These methods include, but are not limited to, oral, duodenal, parenteral injection (including intravenous, subcutaneous, intraperitoneal, intramuscular, intraarterial injection or infusion), topical administration, and rectal administration. The techniques of the administration of the compounds and methods described herein are well known to those skilled in the art, for example, those discussed in Goodman and Gilman, The Pharmacological Basis of Therapeutics, current ed.; Pergamon; and Remington's, Pharmaceutical Sciences (current edition), Mack Publishing Co., Easton, Pa. In a preferred embodiment, the compounds and compositions discussed herein are administered orally.

In the present application, the terms “pharmaceutical combination”, “drug combination”, “combination”, “administering other treatments”, “administering other therapeutic agents” and the like mean a pharmaceutical treatment obtained by mixing or combining more than one active ingredient, including both fixed and unfixed combinations of active ingredients. The term “fixed combination” refers to simultaneous administrating at least one compound described herein and at least one synergistic agent to a patient in the form of a single entity or a single dosage form. The term “unfixed combination” refers to simultaneous administrating, administrating in combination or sequentially administrating in variable interval time at least one of the compounds described herein and at least one synergistic formulation to the patient in the form of separate entities. These can also be applied to a cocktail therapy, for example, administrating three or more active ingredients.

The present invention will be further illustrated below with reference to the specific examples. It should be understood that these examples are only to illustrate the invention but not to limit the scope of the invention. The experimental methods with no specific conditions described in the following examples are generally performed under the conventional conditions, or according to the manufacturer's instructions. Unless indicated otherwise, parts and percentage are calculated by weight.

### Example 1 Experiment of Berberrubine in Combination with Tofacitinib

### 1.1 Materials and Methods

Fifty-one C57 BL/6 male mice (6-10 weeks old, of which the body weight was 20-25 g) were raised by Shanghai Model Organisms Center, Inc. according to the specific pathogen-free (SPF) animal feeding requirements. Oxazolone (4-Ethoxymethy-lene-2-phenyl-2-oxazolin-5-one) was purchased from Sigma; Fecal Occult Blood Diagnostic Kit was purchased from BASO Biology; olive oil was purchased from Adamas Company; and acetone was purchased from Greagent.

Animal model establishment and compound treatment: 51 C57BL/6 mice, of which 3 were used as the normal background controls, and the remaining 48 mice were used to establish an oxazolone-induced colitis model according to the method of Heller et al.. On the first day, the skin on the back of the mice was shaved (2 cm×2 cm), and 150 μl of 3% oxazolone solution (dissolved in a 4:1 mixture of acetone and olive oil) was applied for pre-sensitization. The pre-sensitized mice were observed daily for body weight, activity, and hair color. On the 6^{th} day after pre-sensitization, the mice were randomly assigned into 7 groups: model vehicle group, tofacitinib (2 mg/kg) group, tofacitinib (10 mg/kg) group, berberrubine (2 mg/kg) group, berberrubine (20 mg/kg) group, tofacitinib (2 mg/kg) + berberrubine (2 mg/kg) group and tofacitinib (10 mg/kg) + berberrubine (2 mg/kg) group. There were 8 animals in the model group, and 7 animals in each other group. The corresponding drugs were given by oral gavage every day, while the background control group and model group were given vehicle solvent, and the dosing volume was 10 mL/kg. Intracolonic injection of oxazolone solution was conducted on the 7^{th} day after pre-sensitization. Before intracolonic injection of oxazolone, the mice were fasted for 12 hours and given ad libitum access to water. The mice were anesthetized by isoflurane inhalation, and the activity and breathing were observed to avoid hyperanesthesia. After anesthesia, a catheter with syringe at one end was slowly inserted into the colon through the anus of a mouse at a depth of about 3.5 cm. 50 μL of 1.2% oxazolone solution (dissolved in 50% ethanol) was injected into each mouse of the model group and the treatment group. After indwelling for 20 seconds, the catheter was pulled out, and the mouse was inverted for 30 seconds. The normal background control group was injected with pure water. All mice were fed normally after awakened from anesthesia.

### 1.2 Results and Analysis

Disease activity index (DAI) scoring: The body weight, stool consistency and the bleeding in stool of the mice were monitored every day after the administration, and the DAI scoring was performed according to Table 1. The scoring was conducted for 5 consecutive days. DAI=weight loss score + stool consistency score + rectal bleeding score. Fecal occult blood was detected by semi-quantitative pyramidon method. The DAI scoring standard are shown in Table 1:

TABLE 1
DAI scoring standard

| score | weight loss (%) | stool consistency | bleeding |
|---|---|---|---|
| 0 | none | normal | fecal occult |
| | | | blood (−) |
| 1 | 1%-5% | | |
| 2 | | semi loose stool | fecal occult |
| | | | blood (+) |
| 3 | 6%-10% | | fecal occult |
| | | | blood (++) |
| 4 | 11%-15% | loose stool | gross blood stool |
| 5 | >15% | | |

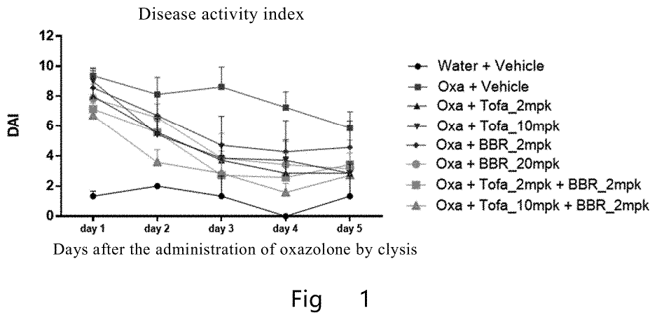

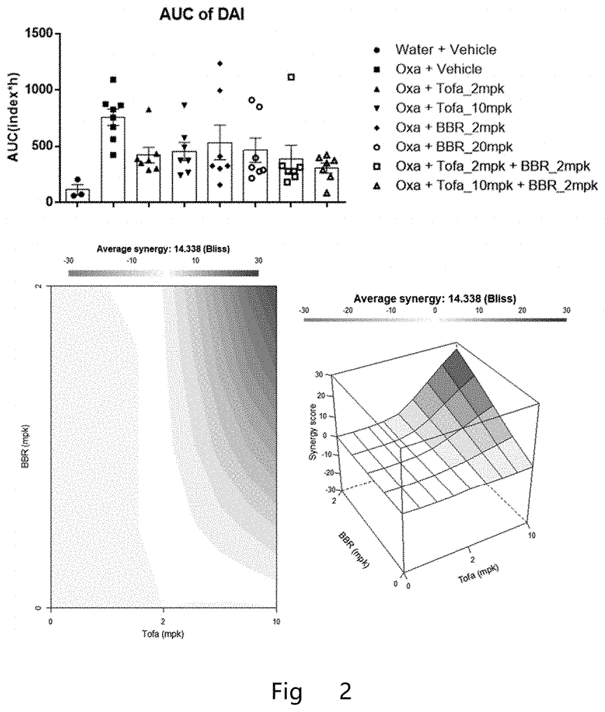

The DAI curve of animal for 5 consecutive days was shown in FIG. 1. The calculated AUC values of DAI for 5 consecutive days and the synergistic effect analysis of the Bliss independent model based on the average value of each group were shown in FIG. 2. The results show that the combination of JAK inhibitor tofacitinib and berberrubine can significantly improve the disease activity index compared with the model vehicle group. And the Bliss independent model synergistic effect analysis based on the mean value showed that, compared with the tofacitinib or berberrubine mono-treatment group, the combination administration group has shown significant synergy effects.

### Example 2 Experiment on the Combination of JAK Inhibitor SHR0302 and Berberrubine

### 2.1 Materials and Methods

The corresponding drugs were given by oral gavage every day, while the background control group and model group were given vehicle, and the dosing volume was 10 mL/kg. Intracolonic injection of oxazolone was conducted on the 7^{th} day after pre-sensitization. Before the intracolonic injection of oxazolone, the mice were fasted for 12 hours and given ad libitum access to water. The mice were anesthetized by isoflurane inhalation, and the activity and breathing were observed to avoid hyperanesthesia. After anesthesia, a catheter with syringe at one end was slowly inserted into the colon through the anus of a mouse at a depth of about 3.5 cm. 50 μL of 1.2% oxazolone solution (dissolved in 50% ethanol) was injected into each mouse of the model group and the treatment group. After indwelling for 20 seconds, the catheter was pulled out, and the mouse was inverted for 30 seconds. The normal background control group was injected with pure water. All mice were fed normally after awakened from anesthesia.

### 2.2. Results and Analysis

Disease Activity Index (DAI) Scoring: The body weight, stool consistency and the presence of blood in stool of the mice were observed every day after the administration, and the DAI scoring was performed according to Table 1. The scoring was conducted for 5 consecutive days. DAI=weight loss score + stool consistency score + rectal bleeding score. Fecal occult blood was detected by semi-quantitative pyramidon method. The DAI scoring standard are shown in Table 1.

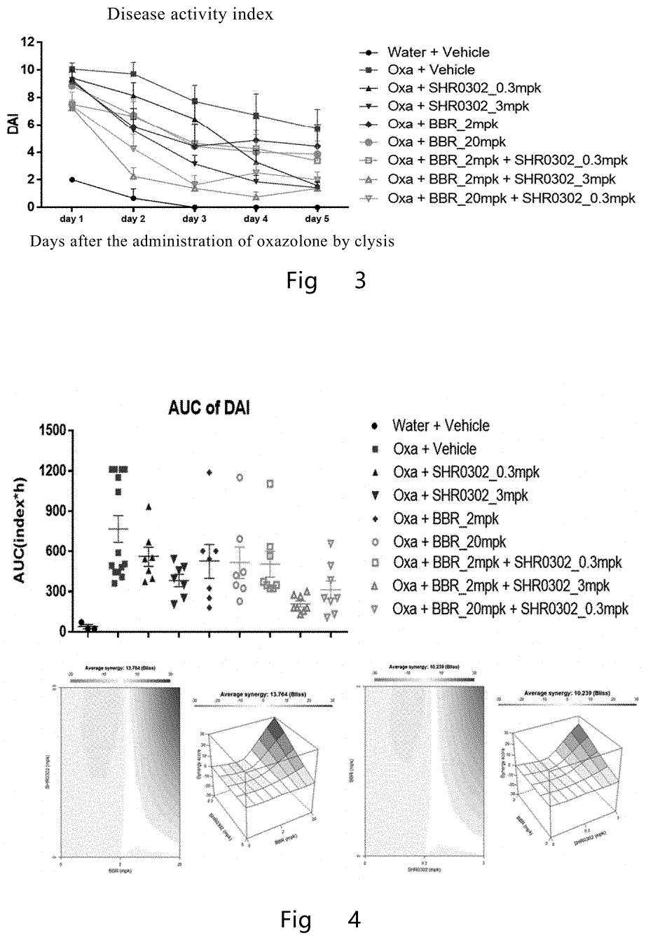

The DAI curve of animal for 5 consecutive days was shown in FIG. 3. The calculated AUC values of DAI for 5 consecutive days and the synergistic effect analysis of the Bliss independent model based on the average value of each group were shown in FIG. 4. The results show that the combination of JAK inhibitor SHR0302 and berberrubine can significantly improve the disease activity index compared with the model group. And the Bliss independent model synergistic effect analysis based on the mean value showed that, compared with the mono-treatment groups, the combination administration group has shown significant synergy effects.

All literatures mentioned in the present application are incorporated herein by reference, as though each one is individually incorporated by reference. Additionally, it should be understood that after reading the above teachings, those skilled in the art can make various changes and modifications to the present invention. These equivalents also fall within the scope defined by the appended claims.

1. A pharmaceutical composition, which comprises:
(A) a therapeutically effective amount of the first active ingredient, wherein the first active ingredient is a berberine analog which has a structure selected from the group consisting of: 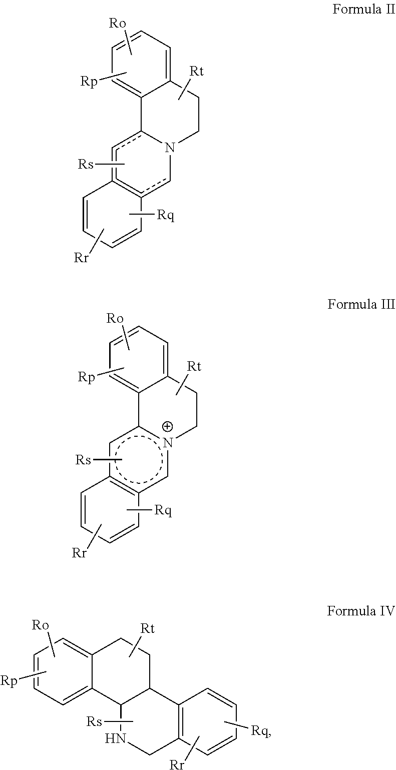
wherein,
dashed lines are chemical bonds or none;
Ro, Rp, Rq, Rr, Rs and Rt are each independently selected from the group consisting of H, hydroxy, substituted or unsubstituted C1-C4 alkyl, substituted or unsubstituted C1-C4 alkoxy;
or two of Ro, Rp, Rq, Rr, Rs and Rt on the same atom together form an oxygen atom;
or Ro, Rp, Rq, Rr, Rs and Rt on two adjacent atoms together with the atoms to which they are attached form a 5-7 membered heterocycle;
wherein the “substituted” means that one or more H atoms on the group are replaced by substituents selected from the group consisting of halogen, C1-C4 alkyl, and phenyl;
(B) a therapeutically effective amount of a second active ingredient, which is a JAK inhibitor;
and the mass ratio of the first active ingredient to the second active ingredient is 1-1000:1000-1,
provided that, when the first active ingredient is berberine, the second active ingredient is other than Tofacitinib.

2. The composition of claim 1, wherein the berberine analog is selected from the group consisting of:

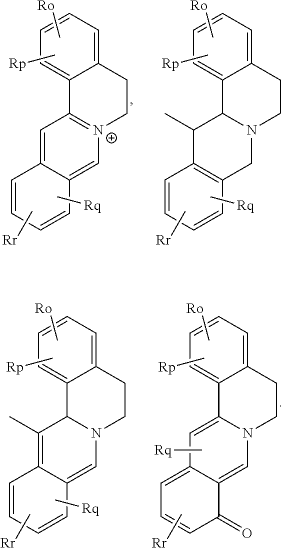

3. (canceled)

4. The composition of claim 1, wherein the berberine analog is selected from the group consisting of:

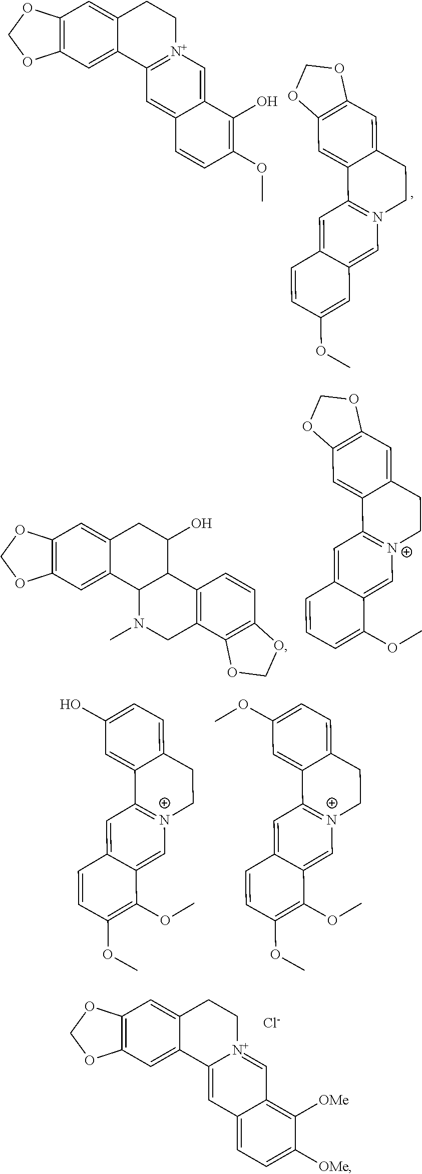

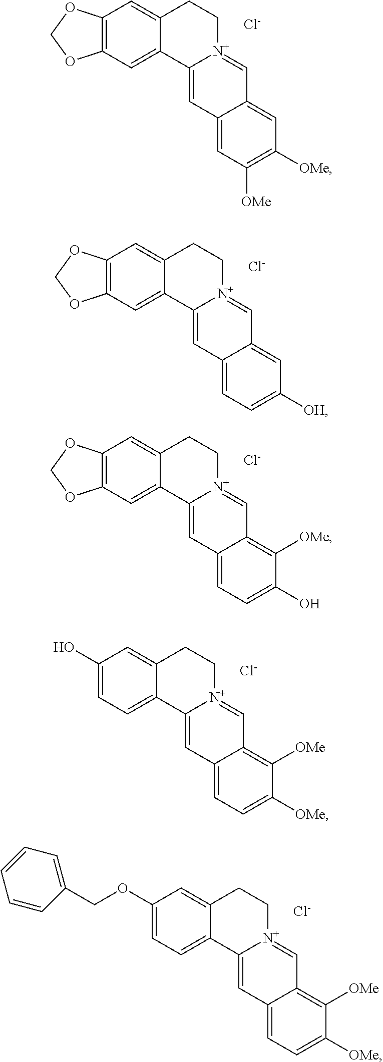

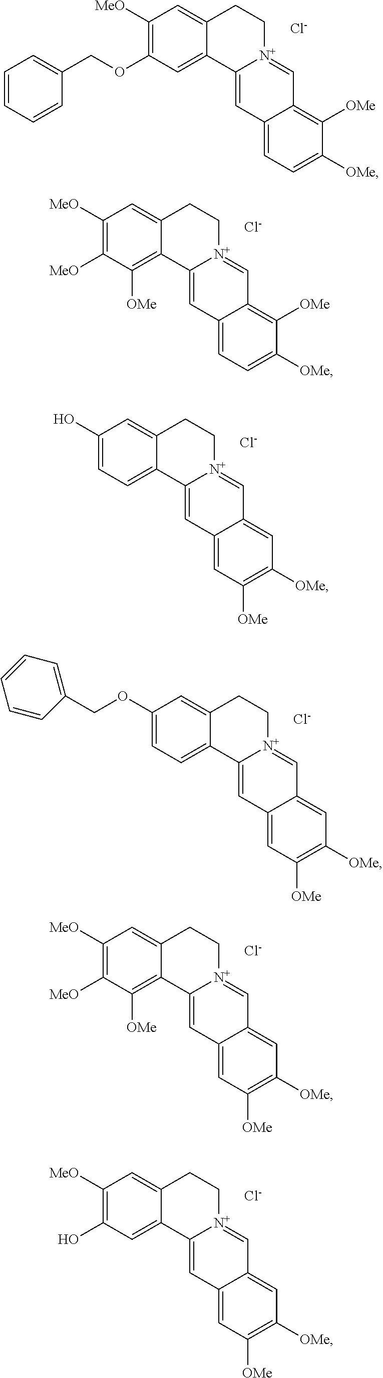

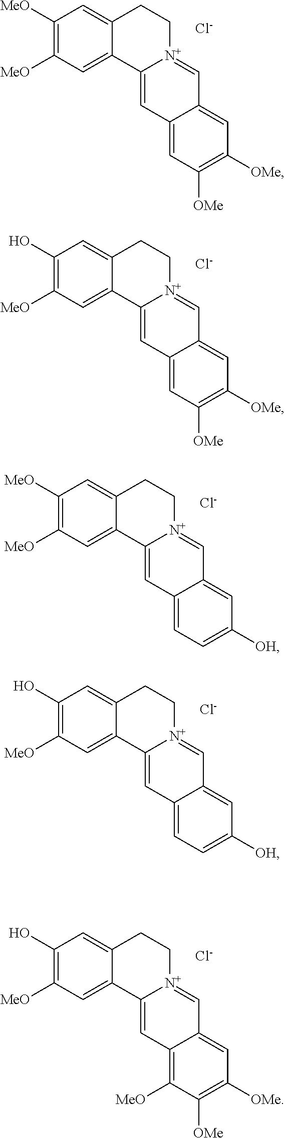

5. The composition of claim 1, wherein the JAK inhibitor can be selected from the group consisting of Tofacitinib, Ruxolitinib, Oclacitinib, Baricitinib, Peficitinib, Abrocitinib, Filgotinib, Upadacitinib , Delgocitinib, Itacitinib, Fedratinib, Decernotinib, SHR-0302, AZD-4205, ASN-002, BMS-986165, PF-06700841, PF-06651600, R-348, INCB-52793, ATI-501, ATI-502, NS-018, KL-130008, deuterium substituted JAK inhibitors, etc.

6. The composition of claim 1, wherein the JAK inhibitor is selected from the group consisting of:

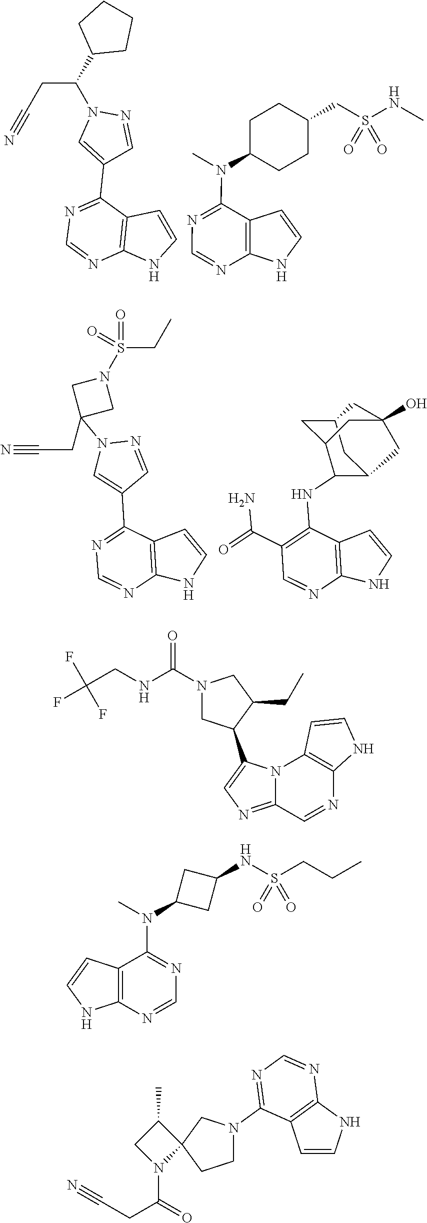

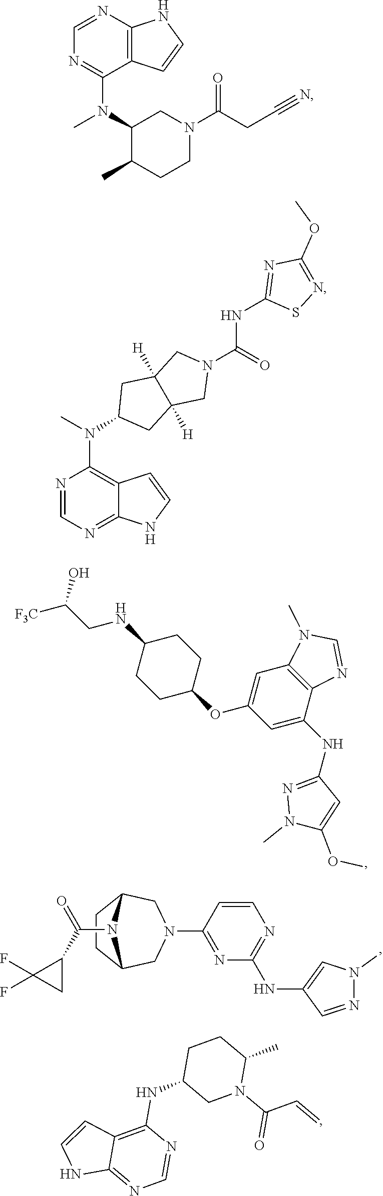

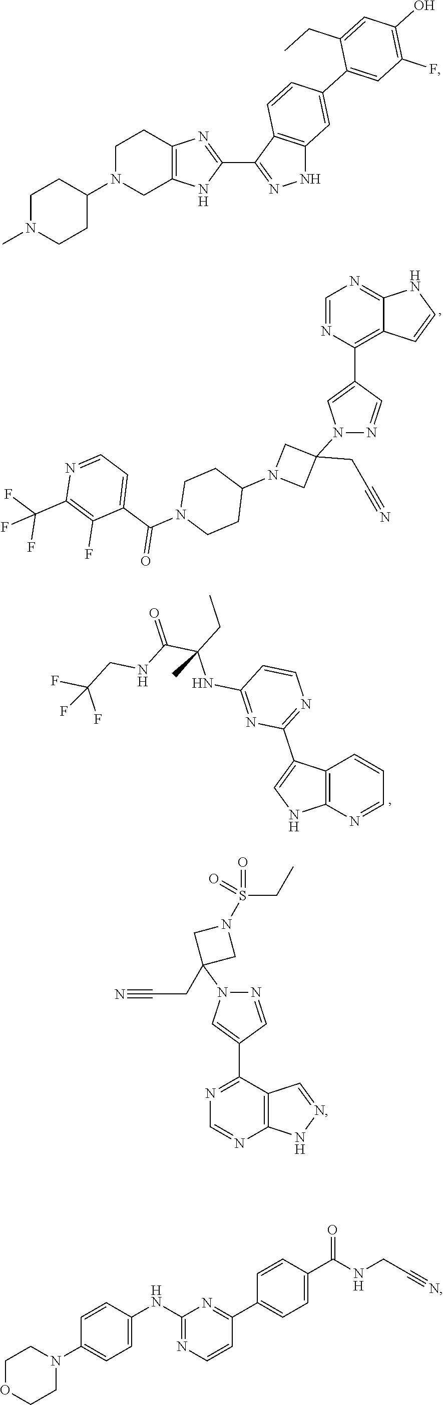 [SMILES: CCc1cc(O)c(F)cc1-c1ccc2c(-c3nc4c([nH]3)CN(C3CCN(C)CC3)CC4)n[nH]c2c1 ; N#CCC1(n2cc(-c3ncnc4[nH]ccc34)cn2)CN(C2CCN(C(=O)c3ccnc(C(F)(F)F)c3F)CC2)C1 ; CC[C@](C)(Nc1ccnc(-c2c[nH]c3ncccc23)n1)C(=O)NCC(F)(F)F ; CCS(=O)(=O)N1CC(CC#N)(n2cc(-c3ncnc4[nH]ncc34)cn2)C1 ; N#CCNC(=O)c1ccc(-c2ccnc(Nc3ccc(N4CCOCC4)cc3)n2)cc1]

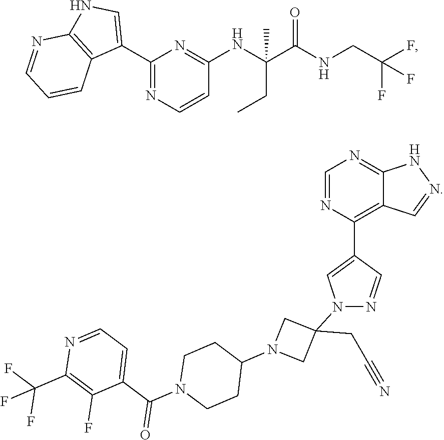 [SMILES: CC[C@@](C)(Nc1ccnc(-c2c[nH]c3ncccc23)n1)C(=O)NCC(F)(F)F ; N#CCC1(n2cc(-c3ncnc4[nH]ncc34)cn2)CN(C2CCN(C(=O)c3ccnc(C(F)(F)F)c3F)CC2)C1]

7. The composition of claim 1, wherein the pharmaceutical composition is enteric-coated preparation.

8. A kit, wherein the kit comprises:
(A) a first formulation comprising a berberine analog;
(B) a second formulation comprising a JAK inhibitor; and
(C) an instruction for use;
and the berberine analog has a structure selected from the group consisting of 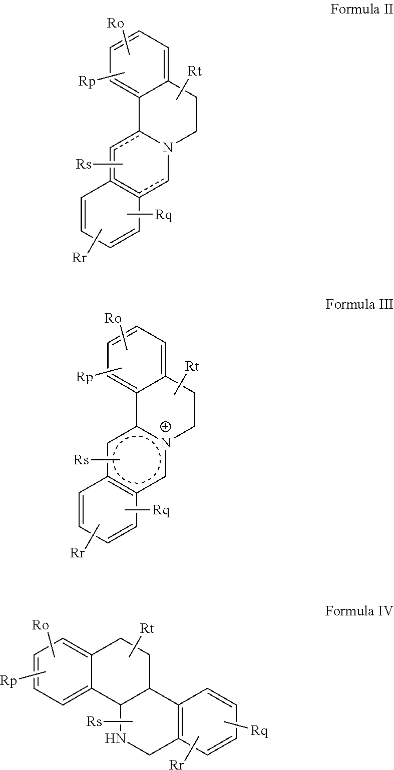
wherein,
dashed lines are chemical bonds or none;
Ro, Rp, Rq, Rr, Rs and Rt are each independently selected from the group consisting of H, substituted or unsubstituted C1-C4 alkyl, substituted or unsubstituted C1-C4 alkoxy;
or Ro, Rp, Rq, Rr, Rs and Rt on two adjacent atoms together with the atoms to which they are attached form a 5-7 membered heterocycle;
wherein the “substituted” means that one or more hydrogen atoms on a group are substituted with a substituent selected from the groups consisting of halogen, C1-C4 alkyl, and phenyl,
provided that, when the first active ingredient is berberine, the second active ingredient is other than Tofacitinib.

9. A combination of active ingredients, wherein the combination comprises following ingredients, or is composed by the following ingredients:
(A) a first active ingredient, wherein the first active ingredient is a berberine analog or a derivative thereof; and
(B) a second active ingredient, which is a JAK inhibitor,
provided that, when the first active ingredient is berberine, the second active ingredient is other than Tofacitinib.

10. A method of treating a disease selected from the group consisting of: Gastrointestinal inflammatory diseases, gastrointestinal autoimmune diseases, peptic ulcer, irritable bowel syndrome, gastric cancer, esophageal cancer, and colon cancer, comprising administrating to a subject in need thereof,
(A) a therapeutically effective amount of a first active ingredient, wherein the first active ingredient is a berberine analog or a derivative thereof; and
(B) a therapeutically effective amount of a second active ingredient, which is a JAK inhibitor,
provided that, when the first active ingredient is berberine, the second active ingredient is other than Tofacitinib.

11. The method of claim 10, wherein the disease is selected from the group consisting of ulcerative colitis, Crohn's disease, colitis associated with immune checkpoint inhibitor therapy, collagenous colitis, lymphocytic colitis, pouchitis, acute/chronic gastritis, acute/chronic appendicitis, graft-versus-host disease, celiac sprue, and autoimmune bowel disease.

12. The composition of claim 1, wherein the mass ratio of the first active ingredient to the second active ingredient is 1-10:10-1.
